(19) United States
(12) Patent Application Publication
(10) Pub. No.: US 2022/0351809 A1
(43) Pub. Date: Nov. 3, 2022

(54) Device and Method for Predicting Product Properties of Naphtha Splitting Unit

(71) Applicant: SK Incheon Petrochem Co., Ltd., Incheon (KR)

(72) Inventors: Min Ho Kim, Incheon (KR); Jung Gon Kim, Incheon (KR); Chang Mook Kim, Incheon (KR); Hyun Joo Kim, Seongnam-si (KR); Ho Dong Park, Seongnam-si (KR)

(21) Appl. No.: 17/732,740

(22) Filed: Apr. 29, 2022

(30) Foreign Application Priority Data
Apr. 30, 2021 (KR) 10-2021-0056216

(51) Int. Cl. G16C 20/30; G16C 20/70; G06N 5/00; G01N 33/28

(52) U.S. Cl. CPC G16C 20/30; G16C 20/70; G06N 5/003; G01N 33/28

(57) ABSTRACT

Provided are a device and method for predicting product properties of a naphtha splitting unit (NSU). The method includes training a prediction model for predicting the product properties of the NSU, inputting an input variable to the trained prediction model to acquire a prediction value for each output variable, and outputting the acquired prediction values for the output variables.

### CROSS-REFERENCE TO RELATED APPLICATION

This application claims priority to Korean Patent Application No. 10-2021-0056216 filed Apr. 30, 2021, the disclosure of which is hereby incorporated by reference in its entirety.

### BACKGROUND OF THE INVENTION

### 1. Field of the Invention

The present disclosure relates to a device and method for predicting product properties of a naphtha splitting unit (NSU). More specifically, the present disclosure relates to a device and method for predicting product properties, which are data that cannot be checked by a chemical process simulator, of an NSU and using the predicted data as operation indicators of the NSU to improve aromatic yield through a follow-up process.

### 2. Description of Related Art

A first process of producing crude oil by mining an oil field and converting the crude oil into transportation fuel, heating fuel, raw materials for petrochemical products, etc. is referred to as an oil refining process. The oil refining process is divided into three processes.

The first process is distillation that is a process of collectively separating components with similar physical and chemical properties using the difference in boiling point between chemical components in the crude oil by raising the temperature of the crude oil which is a mixture of the chemical components. The second process is refining that is a process of improving the quality of a distilled product by removing impurities, which include components that cause air pollution, such as sulfur, contained in a distilled intermediate product. The third process is blending that is a process of mixing refined intermediate products for each product or adding an additive.

A naphtha splitting process, which is a part of a refining process, is performed by a naphtha splitting unit (NSU). A naphtha splitter column of the NSU splits whole straight run (WSR) naphtha, which is feed, into light straight run (LSR) naphtha and heavy straight run (HSR) naphtha.

However, existing chemical process simulators do not have property data about feed of naphtha splitter columns, and thus it is difficult to check the gap between LSR naphtha and HSR naphtha using the chemical process simulators.

Therefore, according to the related art, a product of a naphtha splitter column is measured once a day, and the naphtha splitter column is operated on the basis of an operator's experience.

### SUMMARY OF THE INVENTION

The present disclosure is directed to providing a device and method for predicting product properties, which are data that cannot be checked by a chemical process simulator, of a naphtha splitting unit (NSU) and using the predicted data as operation indicators of the NSU to improve aromatic yield through a follow-up process.

According to an aspect of the present invention, there is provided a method of predicting product properties of an NSU, the method including training a prediction model for predicting the product properties of the NSU, inputting an input variable to the trained prediction model to acquire a prediction value for each output variable, and outputting the acquired prediction values for the output variables.

The training of the prediction model may include generating a random forest model as the prediction model and training the random forest model using, as training data, results of analyzing past operating conditions for a naphtha splitter column of the NSU and product properties acquired under the past operating conditions.

The input variable may include a current operating condition for the naphtha splitter column of the NSU, and the operating condition may include at least one of the amount of middle pressure steam supplied to the naphtha splitter column, the amount of return flow, an operating pressure, and an operating temperature.

The output variables may be variables for determining the product properties of the NSU and include the amount of light straight run (LSR) D95, the amount of LSR D90, the amount of heavy straight run (HSR) D05, and the amount of HSR C6 paraffin.

The method may further include controlling operations of the NSU on the basis of the output prediction values.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a block diagram schematically illustrating oil refining facility;

FIG. 2 is a diagram schematically illustrating a naphtha splitting unit (NSU) shown in FIG. 1;

FIG. 3 is a diagram illustrating a configuration of a device for predicting product properties of an NSU according to an exemplary embodiment of the present disclosure;

FIGS. 4(A) and 4(B) are a set of diagrams illustrating a single decision tree model and a random forest model;

FIG. 5 illustrates prediction results of a prediction unit shown in FIG. 3 and is a graph in which prediction values and measurement values are comparatively shown regarding light straight run (LSR) D95 among product properties of an NSU;

FIG. 6 illustrates prediction results of a prediction unit shown in FIG. 3 and is a graph in which prediction values and measurement values are comparatively shown regarding heavy straight run (HSR) C6 paraffin among product properties of an NSU;

FIG. 7 is a table illustrating root mean square errors (RMSEs) for output variables of a prediction unit;

FIG. 8 is a flowchart illustrating a method of predicting product properties of an NSU according to the exemplary embodiment of the present disclosure;

FIG. 9 is a diagram illustrating a configuration of a device for predicting product properties of an NSU according to another exemplary embodiment of the present disclosure; and

FIG. 10 is a flowchart illustrating a method of predicting product properties of an NSU according to another exemplary embodiment of the present disclosure.

### DESCRIPTION OF THE INVENTION

Advantages and features of the present invention and methods of achieving the same will made clear by referring to exemplary embodiments described in detail with reference to the accompanying drawings. However, the present disclosure is not limited to the exemplary embodiments disclosed herein and may be implemented in various forms. The exemplary embodiments are only provided so that this disclosure of the present invention will be thorough and complete and will fully convey the scope of the present disclosure to those of ordinary skill in the art. The present invention is only defined by the scope of the claims.

Unless otherwise defined, all terms (including technical and scientific terms) used herein have the same meaning as commonly understood by those of ordinary skill in the art. Also, terms defined in commonly used dictionaries will not be interpreted in an idealized or overly formal sense unless clearly so defined herein.

Terms used herein are provided only to describe the exemplary embodiments and not to limit the present disclosure. In this specification, the singular forms include the plural forms as well unless the context clearly indicates otherwise. As used herein, the terms “comprises” and/or “comprising” do not preclude the presence or addition of one or more components other than stated components.

Hereinafter, exemplary embodiments of the present disclosure will be described with reference to the accompanying drawings. Throughout the drawings, like reference numerals refer to like components.

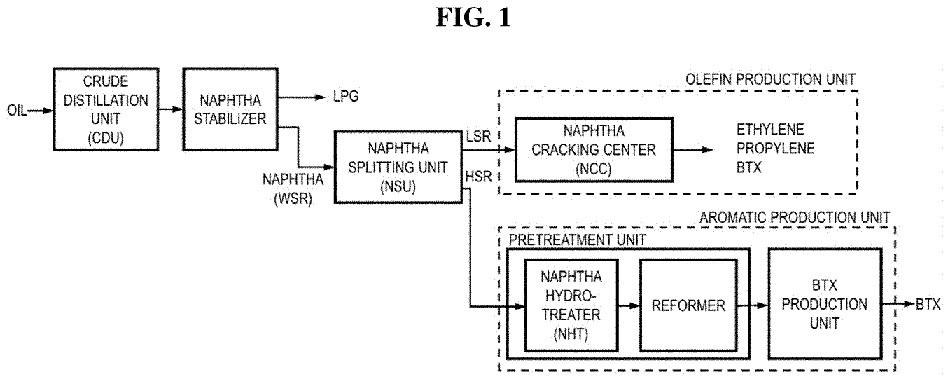

FIG. 1 is a block diagram schematically illustrating oil refining facility.

Referring to FIG. 1, the oil refining facility includes a crude distillation unit (CDU), a naphtha stabilizer, a naphtha splitting unit (NSU), an olefin production unit, and an aromatics production unit.

The CDU is also called an A-tower, an A-column, a topper, or a topping column and is a unit that separates crude oil into liquefied petroleum gas (LPG), naphtha, kerosene, diesel, and bunker C (B-C) oil on the basis of boiling points. LPG and naphtha correspond to light distillates, kerosene and diesel correspond to middle distillates, and B-C oil corresponds to a heavy distillate. This facility is based on the fact that constituents of crude oil have different properties and thus evaporate into gases at certain pressures and temperatures. The temperature and pressure in the column are lower toward the top of the column such that mixed distillates are separated into individual distillates. The CDU is operated at a normal pressure which is similar to atmospheric pressure and thus is called a atmospheric distillation column.

The inside of the atmospheric distillation column includes about 40 to 50 stages, and crude oil is heated to about 350° C. through a heat exchanger and a heater and then inserted thereinto. Each stage is distinguished by a tray installed at every height of about 1 m. Each tray is made of an iron plate in a cap form (bubble cap tray) having a structure in which bubbles are easily generated, a form in which small through holes are drilled in the iron plate, or a form (valve tray plate) in which a valve is installed on the iron plate. Accordingly, liquid components which are condensed in the upper stage and move down smoothly come into contact with gas components which are evaporated in the lower stage and move up such that vapor-liquid equilibrium is easily achieved.

The naphtha stabilizer is a distillation column that only separates whole straight run (WSR) naphtha by removing gas distillations (LPG and fuel gas) from naphtha distillations retrieved from the highest stage of the atmospheric distillation column.

The NSU is a facility that splits the WSR naphtha retrieved from the naphtha stabilizer into light straight run (LSR) naphtha and heavy straight run (HSR) naphtha.

The olefin production unit is a facility that produces base distillates, such as ethylene, propylene, and benzene, toluene, xylene (BTX), using LSR naphtha as a feed. The olefin production unit includes a naphtha cracking center (NCC).

The aromatics production unit is a facility that produces aromatics, such as benzene, toluene, paraxylene, using HSR naphtha as a feed. The aromatics production unit includes a pretreatment unit including a naphtha hydro-treater (NHT) and a reformer and a BTX production unit.

The NHT is a facility that removes sulfur (S), nitrogen (N), and metal present in HSR naphtha and saturates olefin. The sulfur, nitrogen, and metals degrade efficiency of a catalyst input to the reformer and thus are necessarily removed. The NHT adds hydrogen under conditions of high temperature and high pressure and removes the sulfur, nitrogen, and metals using the catalyst. The naphtha from which the sulfur, nitrogen, and metals are removed through the NHT is supplied to the reformer.

The reformer converts naphthene and paraffin components in the naphtha into aromatic components, thereby producing aromatic-rich reformates.

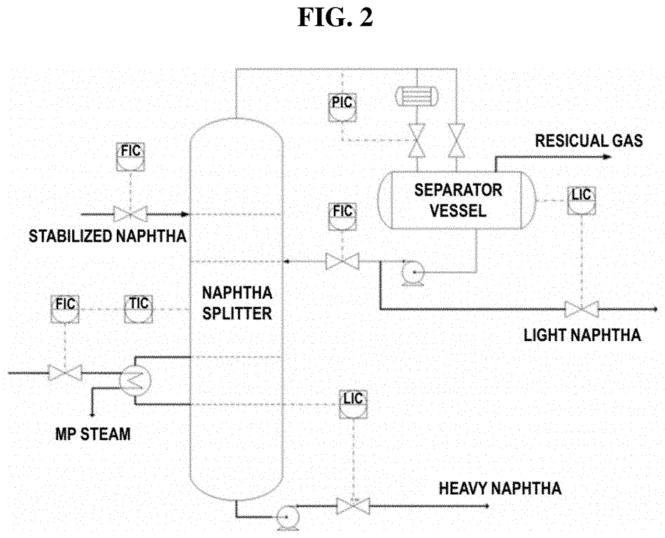

FIG. 2 is a diagram schematically illustrating an NSU shown in FIG. 1.

Referring to FIG. 2, the NSU includes a naphtha splitter column, a heat exchanger, a separator vessel, and control valves separately installed in pipes.

The naphtha splitter column is supplied with stabilized naphtha and medium pressure (MP) steam. The naphtha splitter column is operated under certain operating conditions (e.g., an operating pressure and an operating temperature).

As a result, light naphtha is retrieved from the top of the naphtha splitter column, and heavy naphtha is retrieved from the bottom of the naphtha splitter column. Specifically, the material retrieved from the top of the naphtha splitter column is introduced into the separator vessel. Residual gas is discharged from the top of the separator vessel, and light naphtha is retrieved from the bottom of the separator vessel.

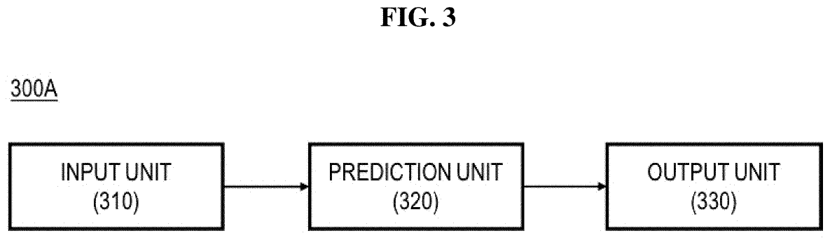

Here, the degree of separation between light naphtha and heavy naphtha varies depending on operating conditions of the naphtha splitter column. Examples of the operating conditions of the naphtha splitter column include the amount of MP steam, the amount of return flow, an operating pressure, and an operating temperature. Since the degree of separation between light naphtha and heavy naphtha varies depending on operating conditions of the naphtha splitter column, when a prediction model is generated using measurement values obtained by measuring light naphtha and heavy naphtha acquired through the naphtha splitter column at certain intervals (e.g., once a day at the same time) and operating conditions of the naphtha splitter column at a time point when the corresponding measurement values are acquired, product properties according to the operating conditions of the naphtha splitter column can be predicted using this prediction model. This will be described in detail with reference to FIG. 3.

FIG. 3 is a diagram illustrating a configuration of a device 300A for predicting product properties of an NSU (hereinafter, “prediction device”) according to an exemplary embodiment of the present invention.

Referring to FIG. 3, the prediction device 300A includes an input unit 310, a prediction unit 320, and an output unit 330.

The input unit 310 receives various pieces of information and/or commands from an operator. For example, the input unit 310 receives current operating conditions of the naphtha splitter column. To this end, the input unit 310 includes an input means, such as a keypad, a touchpad, a touch panel, etc. The touch panel includes a resistive touch panel, a capacitive touch panel, an ultrasonic touch panel, or an infrared touch panel. The touch panel is stacked on a display (not shown) of the output unit 330 to be described below, constituting a touch screen.

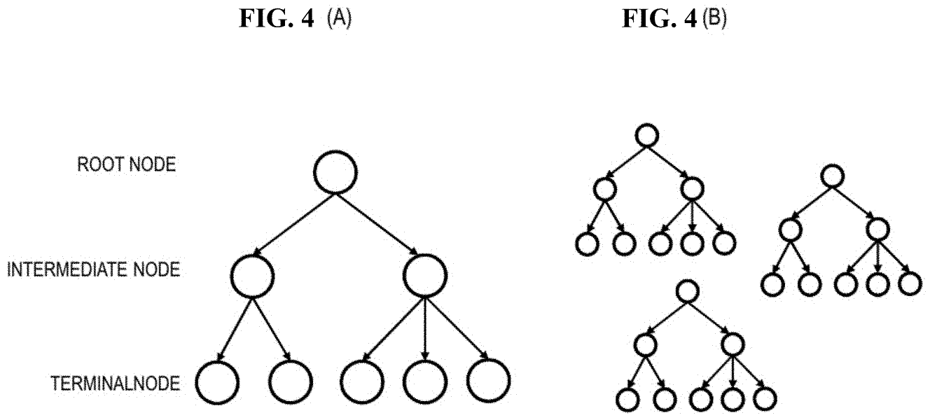

The prediction unit 320 predicts properties of a product produced by the NSU on the basis of the current operating conditions of the naphtha splitter column. To this end, the prediction unit 320 includes a prediction model that has been trained in advance. According to the exemplary embodiment, the prediction model may be trained in advance using a random forest method. Random forest is an ensemble learning method for learning a plurality of decision trees. In ensemble learning methods, a plurality of learning algorithms are used for obtaining better prediction performance than a case of separately using learning algorithms. Decision tree and random forest will be described below with reference to FIG. 4.

FIG. 4(A) illustrates a single decision tree model, and FIG. 4(B) illustrates a random forest model.

Referring to FIG. 4(A), the single decision tree model includes a root node, intermediate nodes, and terminal nodes or leaf nodes. The root node is a initial points, and as branch are repeated the number of pieces of data corresponding to each branch decreases. The sum of the numbers of pieces of data belonging to the terminal nodes corresponds to the number of pieces of data of the root node. This denotes that there is no intersection between the terminal nodes. FIG. 4(A) illustrates a case in which there are five terminal nodes, and the five terminal nodes denote that all the data is divided into five subsets. The single decision tree allows both classification and regression.

Referring to FIG. 4(B), the random forest model includes a plurality of decision trees. The random forest model allows classification with high precision, regression, and clustering based on collective learning. As shown in FIG. 4(A), machine learning is allowed by a single decision tree alone. However, a single decision tree has a drawback in that it tends to be overfitted to training data. Therefore, when a random forest is made up of several decision trees as shown in FIG. 4(B), it is possible to overcome the drawback of overfitting.

For example, it is assumed that there are 30 features to be considered to predict a result. When a single decision tree is created on the basis of the 30 features, there are a large number of branches, resulting in overfitting. Therefore, only five of the 30 features are randomly selected to create a single decision tree, and then this is repeated to create several decision trees. When the most frequent value among prediction values generated by the several decision trees is designated a final prediction value, it is possible to solve overfitting. In other words, a random forest may be considered as creating several small decision trees rather than creating one huge (deep) decision tree. The random forest allows both classification and regression. Classification is a method of selecting the most frequent one of values predicted by several small decision trees as an output variable. Regression is a method of predicting the average of values predicted by several small decision trees as an output variable.

Referring back to FIG. 3, the prediction unit 320 includes the random forest model described above. In the random forest model, past operating conditions of the naphtha splitter column and results of analyzing properties of products acquired under the operating conditions are used as training data. Examples of variables for determining properties of products include LSR D95, LSR D90, HSR D05, and HSR C6 paraffin. Here, “D” denotes diameter, meaning a particle diameter. More specifically, the minimum size, the maximum size, and the mean size of particles are measured. As a result of measuring a distribution of the particles, a size value corresponding to 90% of the maximum size value in the cumulative distribution is referred to as “D90.” Likewise, a size value corresponding to 95% of the maximum size value in the cumulative distribution is referred to as “D95.”

The random forest model which has completed learning receives current operating conditions of the naphtha splitter column as input variables and outputs the amounts of LSR D95, LSR D90, HSR D05, and HSR C6 paraffin as output variables. The difference between the amount of LSR D95 and the amount of HSR D05 among the output variables is used as a criterion for distinguishing the degree of separation between light naphtha and heavy naphtha. Also, among the output variables, the amount of HSR C6 paraffin is a major factor that affects aromatic yield in a reforming process of the latter part. The lower the value, the higher the aromatic yield.

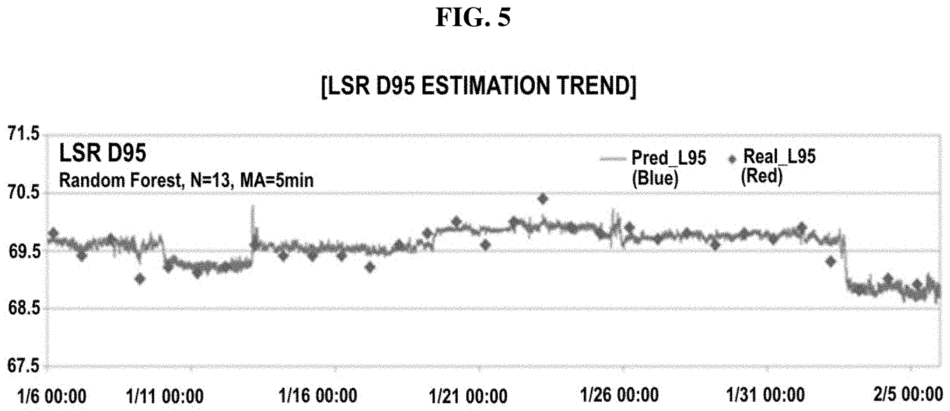

FIG. 5 illustrates prediction results of the prediction unit 320 shown in FIG. 3 and is a graph in which prediction values and measurement values are comparatively shown regarding LSR D95 among product properties of an NSU.

In the graph shown in FIG. 5, the horizontal axis represents time, and the vertical axis represents the amount of LSR D95. Also, the blue solid line represents prediction values output from the prediction unit 320, and the red dots represent measurement values that are actually measured. As shown in FIG. 5, a measurement value of LSR D95 is acquired only once a day, whereas a prediction value of LSR D95 is continuously acquired over time.

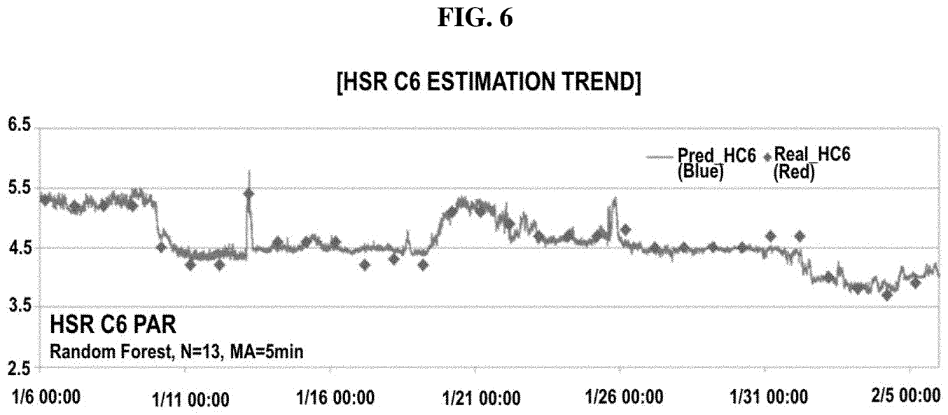

FIG. 6 illustrates prediction results of the prediction unit 320 shown in FIG. 3 and is a graph in which prediction values and measurement values are comparatively shown regarding HSR C6 paraffin among product properties of an NSU.

In the graph shown in FIG. 6, the horizontal axis represents time, and the vertical axis represents the amount of HSR C6 paraffin. Also, the blue solid line represents prediction values output from the prediction unit 320, and the red dots represent measurement values that are actually measured. As shown in FIG. 6, a measurement value of HSR C6 paraffin is acquired only once a day, whereas a prediction value of HSR C6 paraffin is continuously acquired over time.

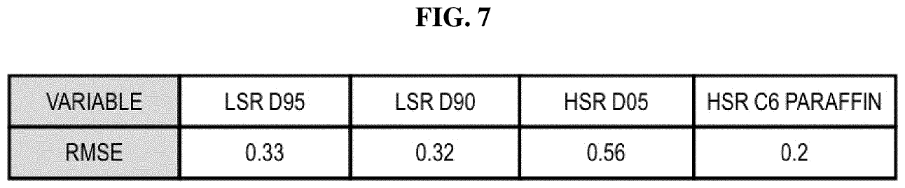

FIG. 7 is a table illustrating root mean square errors (RMSEs) for individual output variables of the prediction unit 320.

An RMSE is obtained by dividing the sum of squares of the value obtained by subtracting the measurement values from the prediction values by the number of samples. In other words, an RMSE is a measure of the difference between a prediction value which is predicted by the prediction unit 320 and a measurement value which is measured in an actual environment. An RMSE is appropriate for representing precision. It may be understood that the smaller the RMSE, the higher the precision.

Referring to FIG. 7, RMSEs of LSR D95, LSR D90, HSR D05, and HSR C6 paraffin are 0.33, 0.32, 0.56, and 0.2, respectively. This denotes that the precision of the random forest model included in the prediction unit 320 is high.

Referring back to FIG. 3, the output unit 330 outputs a command processing result and/or various pieces of data as at least one of a visual signal and an auditory signal. For example, the output unit 330 outputs a prediction value, which is a prediction result of the prediction unit 320, for each output variable as at least one of a visual signal or an auditory signal. To this end, the output unit 330 includes a display (not shown) for outputting visual signals and a speaker (not shown) for outputting auditory signals. The display may be provided in the form of a flat panel display, a flexible display, an opaque display, a transparent display, or electronic paper (E-paper) or any form that is well known in the technical field to which the present disclosure pertains.

When the prediction value for each output variable is output through the output unit 330, the operator may check the output prediction results and then input changed operating conditions for the naphtha splitter column through the input unit 310. For example, the operator may check a prediction value for HSR C6 paraffin among the output prediction values, determine changed operating conditions for the naphtha splitter column to reduce the prediction value for HSR C6 paraffin, and control operations of the naphtha splitter column on the basis of the determined operating conditions.

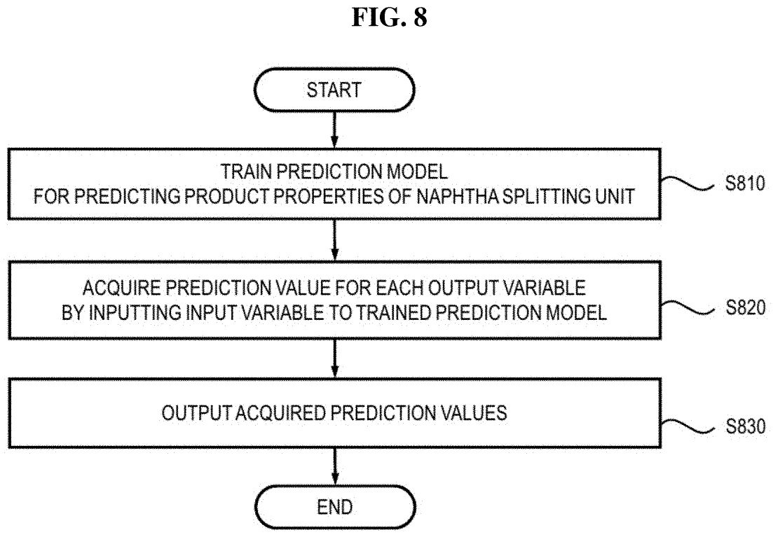

FIG. 8 is a flowchart illustrating a method of predicting product properties of an NSU according to the exemplary embodiment of the present disclosure.

First, a prediction model required for predicting product properties of an NSU is trained (S810). The operation S810 may include an operation of generating a prediction model and an operation of training the prediction model using, as training data, results of analyzing past operating conditions for a naphtha splitter column and properties of products acquired under the past operating conditions. Here, the prediction model may be a random forest model including 13 nodes.

When the training of the prediction model is completed, a prediction value for each output variable is acquired by inputting input variables to the trained prediction model (S820). The input variables include current operating conditions of the naphtha splitter column. Examples of the current operating conditions of the naphtha splitter column include the amount of MP steam, the amount of return flow, an operating pressure, and an operating temperature. The output variable is a variable for determining product properties in the naphtha splitter column, and examples of the output variable include the amount of LSR D95, the amount of LSR D90, the amount of HSR D05, and the amount of HSR C6 paraffin. In other words, as prediction values for output variables, the prediction model outputs a prediction value for the amount of LSR D95, a prediction value for the amount of LSR D90, a prediction value for the amount of HSR D05, and a prediction value for the amount of HSR C6 paraffin.

The prediction values acquired through the prediction model are output as visual signals and/or auditory signals through the output unit 330 (S830).

Subsequently, the operator may control operations of the NSU using the output prediction values as operation indicators. Specifically, the operator may check the output prediction values and then input changed operating conditions for the naphtha splitter column through the input unit 310. Then, operations of the naphtha splitter column may be controlled under the input operating conditions.

The prediction device 300A according to the exemplary embodiment of the present disclosure has been described above with reference to FIGS. 3 to 8. A prediction device 300B according to another exemplary embodiment of the present disclosure will be described below. Descriptions of the same components as shown in FIG. 3 will be omitted, and differences will be mainly described.

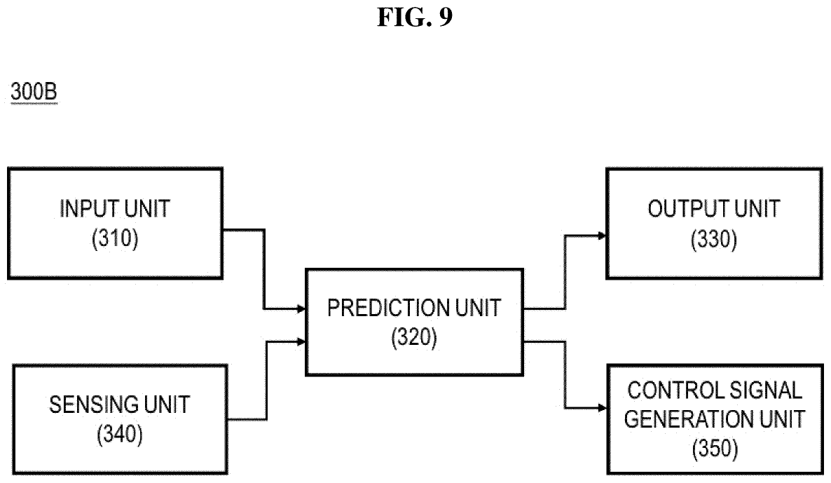

FIG. 9 is a diagram illustrating a configuration of the prediction device 300B according to the other exemplary embodiment of the present disclosure.

Referring to FIG. 9, the prediction device 300B according to the other exemplary embodiment additionally includes a sensing unit 340 and a control signal generation unit 350 compared to the prediction device 300A according to the exemplary embodiment.

The sensing unit 340 is a part that senses current operating conditions of a naphtha splitter column and may include at least one of a steam sensor which senses the amount of MP steam, a flow sensor which senses the amount of return flow, a pressure sensor which senses an operating pressure, and a temperature sensor which senses an operating temperature. A sensing value of each sensor is provided to the prediction unit 320.

The control signal generation unit 350 generates a control signal for controlling operations of the naphtha splitter column on the basis of a prediction value for each output variable which is a prediction result of the prediction unit 320. For example, when a prediction value for the amount of HSR C6 paraffin among the prediction values predicted by the prediction unit 320 is a first reference value or more, a control signal for controlling operations of the naphtha splitter column is generated so that the prediction value for the amount of HSR C6 paraffin may be smaller than or equal to a second reference value which is smaller than the first reference value. For example, the control signal generation unit 350 generates a control signal for controlling at least one of the amount of MP steam, the amount of return flow, an operating pressure, and an operating temperature. Each component of the NSU may be controlled by the generated control signal.

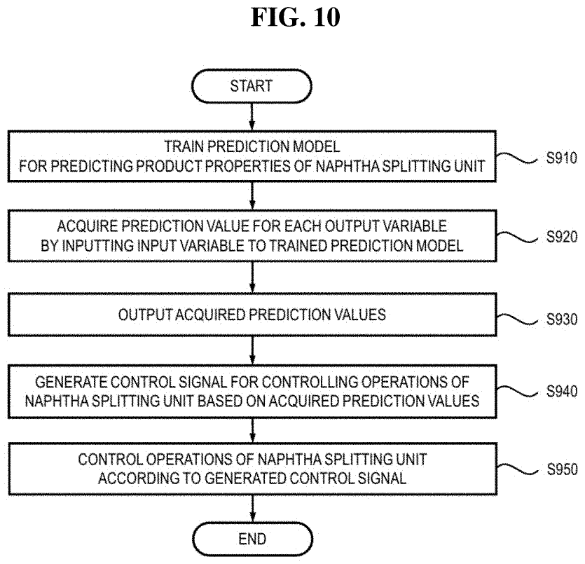

FIG. 10 is a flowchart illustrating a method of predicting product properties of an NSU according to another exemplary embodiment of the present disclosure.

First, a prediction model required for predicting product properties of an NSU is trained (S910). The operation S910 may include an operation of generating a prediction model and an operation of training the prediction model using, as training data, results of analyzing past operating conditions for a naphtha splitter column and product properties acquired under the past operating conditions. Here, the prediction model may be a random forest model including 13 nodes.

When the training of the prediction model is completed, a prediction value for each output variable is acquired by inputting input variables to the trained prediction model (S920).

The input variables include current operating conditions of the naphtha splitter column. Examples of the current operating conditions of the naphtha splitter column include the amount of MP steam, the amount of return flow, an operating pressure, and an operating temperature, and the current operating conditions may be sensed by the sensing unit 340. The output variable is a variable for determining product properties of the naphtha splitter column, and examples of the output variable include the amount of LSR D95, the amount of LSR D90, the amount of HSR D05, and the amount of HSR C6 paraffin. In other words, as output variables, the prediction model outputs a prediction value for the amount of LSR D95, a prediction value for the amount of LSR D90, a prediction value for the amount of HSR D05, and a prediction value for the amount of HSR C6 paraffin.

The prediction values acquired through the prediction model are output as visual signals and/or auditory signals through the output unit 330 (S930). Then, the operator can check the output prediction values in real time.

Meanwhile, the control signal generation unit 350 generates a control signal for controlling operations of the NSU on the basis of the acquired prediction values (S940). For example, a control signal for controlling at least one of the amount of MP steam supplied to the naphtha splitter column, the amount of return flow, an operating pressure, and an operating temperature may be generated.

Subsequently, operations of the NSU are automatically controlled according to the generated control signal (S950).

Exemplary embodiments of the present disclosure have been described above. In the above-described exemplary embodiments, the prediction devices 300A and 300B may be understood as software sensors that generate data by processing data.

Although FIGS. 3 and 9 show the prediction device 300A and 300B as one physical device, this is only for convenience of understanding, and the prediction device 300A and 300B may be implemented as a system including a plurality of computing devices according to an embodiment. For example, the input unit 310, the prediction unit 320, and the output unit 330 may be implemented as separate computing devices. In this case, the computing devices may communicate through a network.

Each of the computing devices may include a storage, which stores an application or computer program for performing a method of predicting product properties of an NSU according to exemplary embodiments of the present disclosure, at least one processor, which performs computation for the application or computer program, a memory, to which the application or computer program performed by the processor is loaded, a bus, which provides a communication function between components of the computing device, and a network interface, which supports wired or wireless Internet communication of the computing device.

In addition to the above-described exemplary embodiments, embodiments of the present disclosure may be implemented through a medium, for example, a computer-readable medium, including computer-readable code or commands for controlling at least one processing element of the above-described exemplary embodiments. The medium may correspond to a medium or media that allow storage and/or transmission of the computer-readable code.

The computer-readable code may be not only recorded in a medium but also transmitted through the Internet. The medium may include, for example, a recording medium, such as a magnetic storage medium (e.g., a read-only memory (ROM), a floppy disk, a hard disk, etc.) or an optical recording medium (e.g., a compact disc (CD)-ROM, a Blu-ray, a digital versatile disc (DVD), etc.) or a transmission medium such as carrier waves. Since the medium may be provided through a distributed network, the computer-readable code may be stored or transmitted and executed in a distributed manner. Further, as an example, the processing element may include a processor or a computer processor and may be distributed and/or included in one device.

When product properties, which are data that cannot be checked by a chemical process simulator, of an NSU are predicted and the predicted data is used as operation indicators of the NSU, it is possible to improve aromatic yield through a follow-up process.

Although embodiments of the present disclosure have been described above with reference to the accompanying drawings, those of ordinary skill in the art should understand that the present disclosure can be implemented in other specific forms without departing from the technical spirit or essential features of the present disclosure. Therefore, it should be understood that the above-described embodiments are exemplary in all aspects and are not limiting.

What is claimed is:

1. A method of predicting product properties of a naphtha splitting unit (NSU), the method comprising the steps:
training a prediction model for predicting the product properties of the NSU;
inputting an input variable to the trained prediction model to acquire a prediction value for each output variable; and
outputting the acquired prediction values for the output variables.

2. The method of claim 1, wherein the training of the prediction model comprises:
generating a random forest model as the prediction model; and
training the random forest model using, as training data, results of analyzing past operating conditions for a naphtha splitter column of the NSU and product properties acquired under the past operating conditions.

3. The method of claim 1, wherein the input variable includes a current operating condition for the naphtha splitter column of the NSU, and
the operating condition includes at least one of an amount of middle pressure steam supplied to the naphtha splitter column, an amount of return flow, an operating pressure, and an operating temperature.

4. The method of claim 1, wherein the output variables are variables for determining the product properties of the NSU and include an amount of light straight run (LSR) D95, an amount of LSR D90, an amount of heavy straight run (HSR) D05, and an amount of HSR C6 paraffin.

5. The method of claim 1, further comprising controlling operations of the NSU on the basis of the output prediction values.

6. A device for predicting product properties of a naphtha splitting unit (NSU), the device comprising:
a prediction unit configured to input, when a prediction model for predicting the product properties of the NSU is trained, an input variable to the trained prediction model and acquire a prediction value for each output variable; and
an output unit configured to output the acquired prediction values for the output variables.

7. The device of claim 6, wherein the prediction model includes a random forest model, and
the prediction model is trained using, as training data, results of analyzing past operating conditions for a naphtha splitter column of the NSU and product properties acquired under the past operating conditions.

8. The device of claim 6, wherein the input variable includes a current operating condition for a naphtha splitter column of the NSU, and
the operating condition includes at least one of an amount of middle pressure steam supplied to the naphtha splitter column, an amount of return flow, an operating pressure, and an operating temperature.

9. The device of claim 6, wherein the output variables are variables for determining the product properties of the NSU and include an amount of light straight run (LSR) D95, an amount of LSR D90, an amount of heavy straight run (HSR) D05, and an amount of HSR C6 paraffin.

10. The device of claim 6, further comprising a control signal generation unit configured to generate a control signal for controlling operations of a naphtha splitter column in the NSU on the basis of the acquired prediction value.

11. A computer-readable recording medium in which a program for performing the method of claim 1 is recorded.

12. An application for terminal devices that is installed on a terminal device, which is hardware, to perform the method of claim 1 and stored in a computer-readable transitory recording medium.
